(19) United States
(12) Patent Application Publication
(10) Pub. No.: US 2021/0179563 A1
(43) Pub. Date: Jun. 17, 2021

(54) PROCESS FOR THE MANUFACTURE OF IMINIUM COMPOUNDS AND THEIR APPLICATION IN THE MANUFACTURE OF PYRAZOLE DERIVATIVES

(71) Applicant: SOLVAY SA, Brussels (BE)

(72) Inventors: Etienne SCHMITT, Soucieu-en-Jarrest (FR); Alexander SCHULZ, Hannover (DE); Janis JAUNZEMS, Hannover (DE)

(21) Appl. No.: 16/771,049

(22) PCT Filed: Dec. 20, 2018
(86) PCT No.: PCT/EP2018/086308

§ 371 Date: Jun. 9, 2020

(30) Foreign Application Priority Data
Dec. 22, 2017 (EP) 17210017.4

(51) Int. Cl. C07D 231/14; C07C 249/16; C07D 231/12

(52) U.S. Cl. CPC C07D 231/14; C07D 231/12; C07C 249/16

(57) ABSTRACT

Process for the manufacture of iminium compounds and their application in the manufacture of pyrazole derivatives The present invention concerns processes for the manufacture of iminium compounds and their application in the manufacture of pyrazole derivatives, in particular in processes for the manufacture of pharmaceutically or agrochemically active compounds.

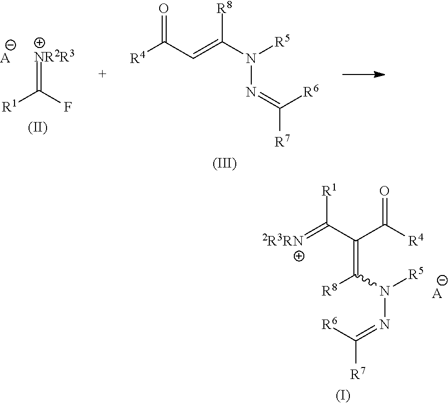

This application claims priority to European application No. 17210017.4, the whole content of this application being incorporated herein by reference for all purposes.

The present invention concerns processes for the manufacture of iminium compounds and their application in the manufacture of pyrazole derivatives, in particular in processes for the manufacture of pharmaceutically or agrochemically active compounds.

Substituted pyrazoles carboxylic acid derivatives, in particular 3-halomethylpyrazole-4-yl carboxylic derivatives, are valuable intermediates in the synthesis of agrochemical and pharmaceutical active ingredients. Agrochemical active ingredients which contain such pyrazole building blocks are, for example, 2′-[1,1′-bicycloprop-2-yl]-3-(difluoromethyl)-1-methylpyrazole-4-carboxanilide (Sedaxane), as described, for example, in WO2006015866, 3-(difluoromethyl)-1-methyl-N-[2-(3′,4′,5′-trifluorophenyl)phenyl]pyrazole-4-carboxamide (Fluxapyroxad), as described, for example, in WO2006087343, N-(3′,4′-Dichloro-5-fluorobiphenyl-2-yl)-3-(difluoromethyl)-1-methylpyrazole-4-carboxamide (Bixafen), as described, for example, in WO2003070705, 3-(Difluoromethyl)-1-methyl-N-[1,2,3,4-tetrahydro-9-(1-methylethyl)-1,4-methanonaphthalen-5-yl]-1H-pyrazole-4-carboxamide (Isopyrazam), as described, for example, in WO2004035589, (RS)—N-[9-(Dichloromethylen)-1,2,3,4-tetrahydro-1,4-methanonaphthalin-5-yl]-3-(difluoromethyl)-1-methyl-1H-pyrazole-4-carboxamide (Benzovindiflupyr), as described, for example, in WO07048556.

Substituted pyrazoles carboxylic acid derivatives, in particular 3-halomethylpyrazole-4-yl carboxylic derivatives, can be obtained by cyclization of dicarbonyl derivatives with subsequent conversion into the free acid, as described for example in WO2017129759 or WO2016152886. Problems which often occur are creation of larger amounts of waste, such as in WO2016152886, where the use of high amounts of hypohalogenous acids for oxidative formation is unfavourable, insufficient selectivity in cyclization either by insufficient distinction of a hydrazine reagent bearing two possible reaction sites or by insufficient distinction of two potential sites for the nucleophilic attack in the dicarbonyl intermediate. Another challenge can be the access to intermediates. It had now been found that the process according to the present invention overcomes the problems mentioned above and thus provides an improved process for the manufacturing of substituted pyrazoles carboxylic acid derivatives.

The present invention thus concerns a process for manufacturing a compound according to formula (I), which comprises the reaction a compound of formula (II) and a compound of formula (III)

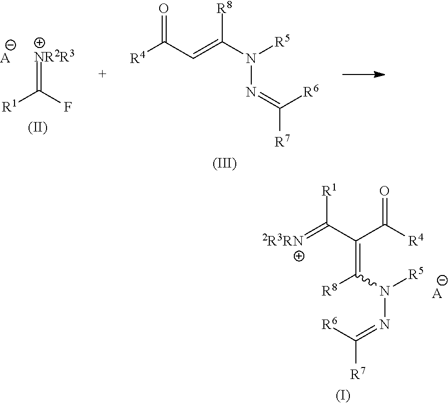

wherein R¹, R², R³, R⁴, R⁵, R⁶, R⁷, R⁸ and A⁻ will be defined in the subsequent description. The invention further concerns processes for the manufacture of compounds of formula (IV), (VII) and (VIII), which will be further defined in the subsequent definition, which comprises the process for the manufacture of the compound of formula (I).

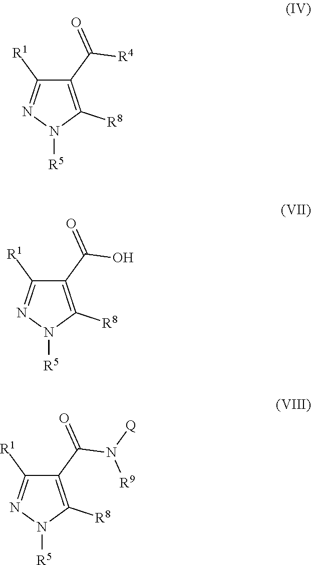

A process for the manufacture of agrochemically and pharmaceutically active compounds comprising at least the process for the manufacture of the compound of formula (I), and optionally at least one process for the manufacture of compound (IV), (VII) and/or (VIII) is a further object of the present invention.

In the present invention, designations in singular are in intended to include the plural; for example, “a solvent” is intended to denote also “more than one solvent” or “a plurality of solvents”.

All aspects and embodiments of the present invention are combinable.

In the context of the present invention, the term “comprising” is intended to include the meaning of “consisting of”.

When a double bond is depicted in a particular E/Z geometry, this is intended to also denote the other geometric form as well as mixtures thereof. Compounds bearing one or more stereocenters are intended to include all mixtures of the stereoisomers, including stereochemically pure isomers.

The invention concerns in a first aspect a process for manufacturing a compound according to formula (I), which comprises the reaction a compound of formula (II) and a compound of formula (III)

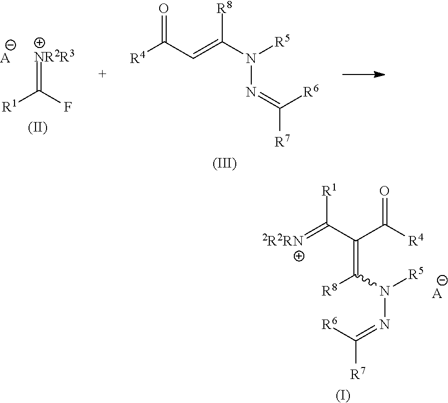

wherein R¹ is selected from the group consisting of C₁₋₄ alkyl groups which is substituted by at least one halogen atom,

wherein R² and R³ are independently selected from the group consisting of C₁-C₁₂-alkyl, C₃-C₁₀-cycloalkyl, aryl, and aralkyl groups, each of which is optionally substituted, or wherein R² and R³ together with the nitrogen atom to which they are bound form an optionally substituted 5- to 10-membered heterocyclic radical which, in addition to the nitrogen atom to which they are attached, may contain a further 1, 2 or 3 heteroatoms selected from the group consisting of O, N and S as ring members,

wherein R⁴ is selected from the group consisting of CF₃, CCl₃ and CBr₃,

wherein R⁵ is selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted,

wherein R⁶ and R⁷ each independently are selected from the group consisting of H, C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted, wherein at least one of R⁶ and R⁷ is different from H

wherein R⁸ is selected from the group consisting of H, X′, COOR′, OR′, SR′, C(O)NR′₂, wherein the groups R′ are selected independently in C(O)NR′₂ where R′ is selected from the group consisting of hydrogen, C₁-C₁₂-alkyl group, CN, C₂-C₆ alkenyl, aryl, cycloalkyl, aralkyl, heteroaryl, each of which is optionally substituted, and wherein X′ is a halogen atom.

and wherein A⁻ is selected from the group consisting of Cl⁻, BF₄⁻, PF₆⁻, SbF₆⁻, AlCl₃F⁻ and AlCl₄⁻.

In the context of the present invention, the term “C₁-C₁₂-alkyl groups” is intended to denote straight or branched alkyl groups having one to twelve carbon atoms. The group comprises, for example, n-nonyl and its isomers, n-decyl and its isomers, n-undecyl and its isomers and n-dodecyl and its isomers, methyl, ethyl, n-propyl, isopropyl, n-, iso-, sec- and t-butyl, n-pentyl and its isomers, n-hexyl and its isomers, 1,3-dimethylbutyl, 3,3-dimethylbutyl, n-heptyl and its isomers and n-octyl and its isomers. Often, the C₁ to C₄ alkyl groups are the most preferred groups of the C₁-C₁₂ alkyl group. The term “C₁-C₄-alkyl group” is intended to denote straight or branched alkyl groups having one to four carbon atoms. This group comprises methyl, ethyl, n-propyl, isopropyl, n-, iso-, sec- and t-butyl. Generally, an alkyl group can optionally be substituted by one or more substituents of the group S*, wherein S* consists of R′, —X′, —OR′, —SR′, —NR′₂, —SiR′₃, —COOR′, —(C═O)R′, —CN and —CONR′₂, wherein R′ is selected independently from the group consisting of hydrogen and C₁-C₁₂-alkyl groups and X′ is selected from the group consisting of F, Cl, Br, or I. R¹, which in its broadest definition is selected from the group consisting of C₁₋₄ alkyl groups which is substituted by at least one halogen atom, preferably is selected from the group consisting of C₁₋₄ alkyl groups which is substituted by at least one fluorine atom. More preferably, R¹ is an ethyl or methyl group which is substituted by at least one fluorine atom and optionally by one or more substituents of the group S* as defined above. This definition includes, for example, CH₂F, CF₃, CCl₂F, CBr₂H, CF₂H, CCl₂H, CHFCl, CHFCF₃ and CHFOCF₃. R¹ even more preferably is a methyl group which is substituted by at least one fluorine atom and optionally by one or more substituents of the group S* as defined above. In a more preferred aspect, R¹ is CF₂H or CF₃, wherein CF₂H is most preferred.

The term “C₂-C₆ alkenyl” intends to denote a group comprising a carbon chain and at least one double bond. Alkenyl group are, for example, ethenyl, propenyl, butenyl, pentenyl or hexenyl. Generally, a C₂-C₆ alkenyl group can optionally be substituted by one or more substituents of the group S* as defined above.

The term “C₃-C₁₀-cycloalkyl” intends to denote mono-, bi- or tricyclic hydrocarbon groups comprising 3 to 10 carbon atoms, in particular 3 to 6 carbon atoms. Examples of monocyclic groups include cyclopropyl, cyclobutyl, cyclopentyl, cyclohexyl, cycloheptyl or cyclooctyl. Examples of bicyclic groups include bicyclo[2.2.1]heptyl, bicyclo[3.1.1]heptyl, bicyclo[2.2.2]octyl and bicyclo[3.2.1]octyl. Examples of tricyclic groups are adamantyl and homoadamantyl. Generally, a C₃-C₁₀-cycloalkyl group can optionally be substituted by one or more substituents of the group S* as defined above.

In the context of the present invention, C₂₋₁₂ alkynyl groups are, unless defined otherwise, straight-chain, branched or cyclic hydrocarbon groups which contain at least one double unsaturation (triple bond) and may optionally have one, two or more single or double unsaturations or one, two or more heteroatoms selected from the group consisting of O, N, P and S. Generally, a C₂₋₁₂-alkynyl group can optionally be substituted by one or more substituents of the group S* as defined above. The definition C₂₋₁₂-alkynyl comprises the largest range defined herein for an alkynyl group. Specifically, this definition comprises, for example, the meanings ethynyl (acetylenyl); prop-1-inyl and prop-2-inyl.

The term “aryl group” intends to denote C₅-C₁₈ monocyclic and polycyclic aromatic hydrocarbons with 5 to 18 carbon atoms in the cyclic system. Specifically, this definition comprises, for example, the meanings cyclopentadienyl, phenyl, cycloheptatrienyl, cyclooctatetraenyl, naphthyl and anthracenyl. Generally, an aryl group can optionally be substituted by one or more substituents of the group S* as defined above.

The term “heteroaryl group” intends to denote C₅-C₁₈ monoyclic and polycyclic aromatic hydrocarbons with 5 to 18 carbon atoms in the cyclic system, wherein one or more methine (—C═) and/or vinylene (—CH═CH—) groups are replaced by trivalent or divalent heteroatoms, in particular nitrogen, oxygen and/or sulphur, respectively, in such a way as to maintain the continuous 7-electron system characteristic of aromatic systems. Specifically, this definition comprises, for example, the meanings 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl. Generally, a heteroaryl group can optionally be substituted by one or more substituents of the group S* as defined above.

The term “aralkyl” intends to denote alkyl groups which are substituted by aryl groups, which have a C₁-C₈-alkylene chain and which may be substituted in the aryl skeleton or the alkylene chain by one or more heteroatoms selected from the group consisting of O, N, P and S.

The optionally substituted 5- to 10-membered heterocyclic radical which, in addition to the nitrogen atom, may contain a further 1, 2 or 3 heteroatoms selected from the group consisting of O, N and S as ring members, which can be formed when R² and R³ and the nitrogen atom to which the two are attached form such optionally substituted 5- to 10-membered heterocyclic radical, can be, for example, a pyrrolidin radical, a piperidine radical, a hexamethylenimine radical, a morpholine radical or a thiomorpholine radical.

In a preferred aspect, A⁻ is AlCl₃F⁻, BF₄⁻ or AlCl₄⁻. BF₄⁻ is more preferred.

In another preferred aspect, R⁴ is CCl₃.

R² and R³ preferably are the same, are preferably both methyl or ethyl, and are most preferably both methyl. The manufacture of compounds of formula (II) is described, for example, in E. Schmitt et al, Eur. J. Org. Chem. 2015, 6052-6060. When the compound of formula (II) has BF₄⁻ as A⁻, it is preferred that 1,1,2,2-Tetrafluoro-N,N-dimethylethanamine is reacted with BF₃ etherate (BF₃.Et₂O) over BF₃ (gas). It has been observed that the use of (BF₃.Et₂O) compared to BF₃ (gas) can improve the control of the step for the manufacture of compound (I). Immediate cyclization of (I) into (IV) can often be prevented, which is seen as an advantage. In the manufacture of compound (II), BF₃ (preferably in the form of etherate) is preferred over AlCl₃ as reactant for the reaction with 1,1,2,2-Tetrafluoro-N,N-dimethylethanamine.

R₈ preferably is H or X′, wherein F is preferred as X′. The most preferred R⁸ is H.

In a preferred aspect, R₅ is H, CH₃ or optionally substituted benzyl. CH₃ is more preferred.

R⁶ and R⁷ preferably each independently are selected from the group consisting of H, C₁-C₁₂-alkyl and aryl, each of which is optionally substituted by one or more substituent of group S* as defined above, wherein when one of R⁶ and R⁷ is H, the other is not H; i.e. at least one of R⁶ and R⁷ is different from H. In a most preferred aspect, R⁶ and R⁷ both are methyl, or R⁶ is H and R⁷ is optionally substituted phenyl.

In a preferred aspect, the invention concerns the process according to the following scheme:

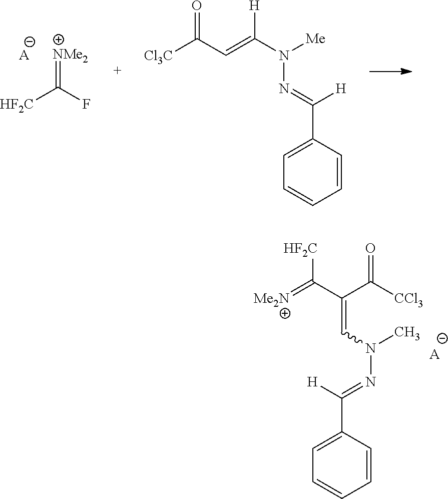

wherein A⁻ is AlCl₃F⁻, AlCl₄⁻ or BF₄⁻.

The compound of formula (III) can be obtained by reacting a compound of formula (X) with a compound of formula (XI)

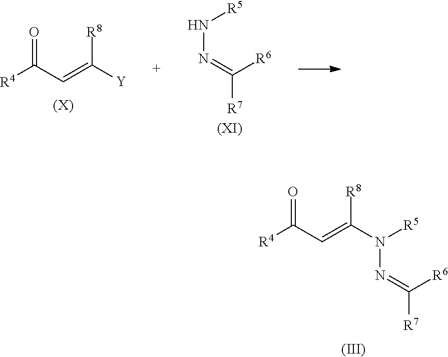

Y is selected from the group consisting of OR¹² or NR¹³R¹⁴, wherein R¹², R¹³ and R¹⁴ are independently selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted, or wherein R¹ and R¹⁴ together with the nitrogen atom to which they are bound form an optionally substituted 5- to 10-membered heterocyclic radical which, in addition to the nitrogen atom, may contain a further 1, 2 or 3 heteroatoms selected from the group consisting of O, N and S as ring members. R⁴, R⁸, R⁵, R⁶ and R⁷ are defined as above. When Y is OR¹², R¹² preferably is methyl or ethyl. When Y is NR¹³R¹⁴, it is preferred that R¹³ and R¹⁴ are methyl, or that R¹³ and R¹⁴ together with the nitrogen atom to which they are bound form a pyrrolidine radical. In a preferred aspect, Y in the compound of formula (X) is NR¹³R¹⁴. The compound of formula (III) wherein Y is NR¹³R¹⁴ can be obtained from a compound of formula (III) wherein Y is OR¹² by reaction with the compound HNR¹³R¹⁴. Such compound are well known in the art, for example, for example 4-ethoxy-1,1,1-trifluoro-3-buten-2-one (ETFBO), which can be obtained through methods described for example in WO2010000871, which is hereby incorporated by reference for all purposes, or (4-ethoxy-1,1,1-trichloro-3-buten-2-one ETCBO), which can be obtained through methods described for example in Tietze, L. F. et al, Organic Syntheses, 69, 238-244; 1990. The hydrazones of the formula (XI) have been described in the literature (Zhurnal Organicheskoi Khimii (1968), 4(6), 986-92.) and can be obtained by reacting commercially available hydrazines with carbonyl compounds. The reaction of the compound of formula (III) wherein Y is NR¹³R¹⁴ with the compound of formula (XI) may advantageously be enhanced by the presence of a catalyst, such as CH₃COOH, H₂SO₄, KHSO₄, HNO₃, H₂PO₄, NaH₂PO₄, HCl, CF₃SO₃H, CF₃COOH and/or CH₃COONa.

The invention also concerns a process for the manufacture of a compound of formula (IV), which comprises the process for the manufacture of compound (I), and which further comprises a step wherein a compound of formula (I) is contacted with an acid

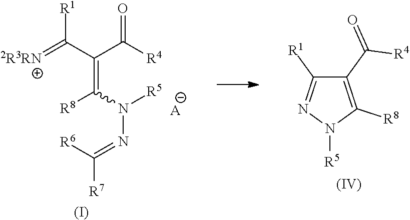

wherein R¹, R², R³, R⁴, R⁵, R⁶, R⁷, R⁸ and A⁻ are defined as above. In a preferred aspect, R¹ is CF₂H, R² and R³ are methyl, R⁴ is CCl₃, R⁵ is methyl, R⁸ is H, R⁶ and R⁷ are methyl, or R⁶ is H and R⁷ is phenyl, and A⁻ is AlCl₃F⁻, AlCl₄⁻ or BF₄⁻. The conditions are selected such that cyclization is achieved in addition to cleavage of the hydrazone moiety. The acid in the manufacture of the compound of formula (IV) from the formula (I) generally is selected from the group consisting of CH₃COOH, H₂SO₄, KHSO₄, HNO₃, H₂PO₄, NaH₂PO₄, HCl, CF₃SO₃H, CF₃COOH and CH₃COONa. The acids can be applied in the presence of water or in the substantial absence of water in their non-aqueous form. For example, aqueous diluted acids may lead to intermediate compounds of formula (V) which will be explained below; for the intended cyclization of (I) to yield (IV) directly, it can be preferred to use aqueous acids of a normality of 1N or higher. Concentrated acids, such as conc. H₂SO₄, are preferred. The temperature in the reaction of the compound of formula (I) with an acid generally is selected from 0° C. to 100° C., preferably from 20° C. to 80° C., more preferably from 20° C. to 70° C.

In another aspect, the invention concerns a process for the manufacture of a compound of formula (IV), which comprises the process for the manufacture of a compound of formula (I), which comprises a step of contacting the compound of formula (I) with water in the presence of an acid or a base under reaction conditions which allow the compound of formula (V) to be formed, followed by a step wherein the compound of formula (V) is contacted with an acid to form the compound of formula (IV), wherein R¹, R², R³, R⁴, R₅, R⁶, R⁷, R⁸ and A⁻ in (I), (V) and (VI), where present, are defined as above.

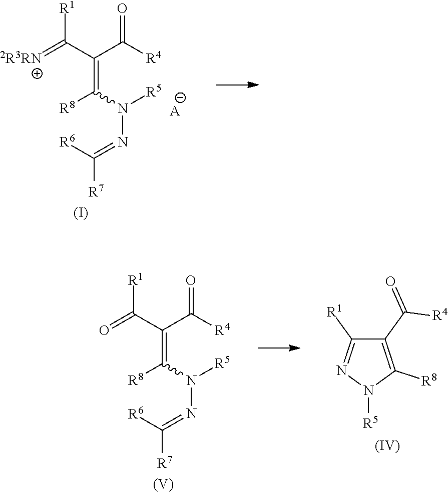

The acid in the manufacture of the compound of formula (V) from the formula (I) generally is selected from the group consisting of CH₃COOH, H₂SO₄, KHSO₄, HNO₃, H₂PO₄, NaH₂PO₄, HCl, CF₃SO₃H, CF₃COOH and CH₃COONa. It can be preferred to use an aqueous acid for the conversion of compound (I) into (V), in particular an aqueous acid of 1N or less. When a base is used in the step of converting the compound of formula (I) into the compound of formula (V), the base in the manufacture of the compound of formula (V) from the formula (I) generally is selected from the group consisting of inorganic bases, such as sodium carbonate, sodium bicarbonate, caesium carbonate, sodium hydroxide, potassium hydroxide, caesium hydroxide, and organic bases such as triethlyamine and pyridine. The amount of acid or base used is preferably 0.8-5.0 eq based on the amount of compound (I), more preferably 0.9-4.0 eq and further preferably 1.0-2.0 eq. The amount of water used is not particularly limited and is preferably 0.5-20 times (wt %) that of compound (I) and more preferably 0.8-5 times (wt %).

Another object of the present invention is a process for the manufacture of a compound of formula (VII), which comprises the process for the manufacture of the compound of formula (IV), and which further comprises a step wherein the compound of formula (IV) is contacted with a base, wherein R¹, R⁴, R₅ and R⁸ are defined as above.

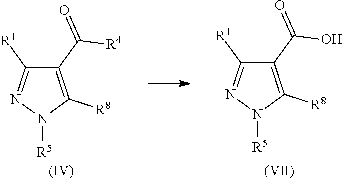

The base in the manufacture of the compound of formula (VII) from the formula (IV) generally is selected from the group consisting of inorganic bases, such as sodium carbonate, sodium bicarbonate, caesium carbonate, sodium hydroxide, potassium hydroxide, caesium hydroxide, and organic bases such as triethlyamine and pyridine. The inorganic bases preferably are used in an aqueous medium. The compound of formula (VI) is generally obtained as the carboxylate anion of formula (VI), wherein the countercation is the cation of the complementary base cation, such as Na⁺ or K⁺.

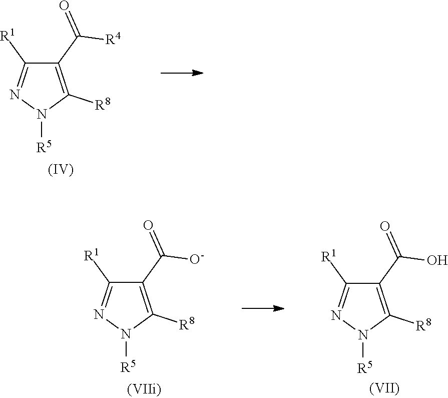

In order to obtain the compound of formula (VII) from the compound of formula (IV), after the compound of formula (IV) is contacted with a base, often the intermediate (VI) is contacted with at least one acid in order to obtain the compound of formula (VII). The at least one acid used for the conversion of the compound of formula (VI) into the compound of formula (VII) is an acid which has sufficient acidity to generate a carboxylic acid, for example, inorganic acids such as hydrochloric acid, sulphuric acid, nitric acid, phosphoric acid and organic acids such as trifluoroacetic acid, methane sulphonic acid and para-toluene sulphonic acid. The amount of acid generally is determined as to set the reaction mixture comprising the compound of formula (VII) and/or (VI) to equal to or lower than pH 5, or preferably equal to or lower than pH 3. The compound of formula (VII) generally is isolated and purified by solvent extraction, distillation, sublimation, crystallisation, silica gel column chromatography, preparative thin layer chromatography, preparative liquid layer chromatography and/or solvent washing, wherein extraction with an organic solvent, evaporation of the solvent and subsequent washing/recrystallization is preferred. Solvents used in isolation and purification selected, for example, from aliphatic halogenated solvents such as dichloromethane, chloroform and 1,2-dichloroethane; aromatic hydrocarbon-type solvents such as benzene, toluene, xylene and anisole; ether-type solvents such as diethyl ether, t-butylmethyl ether, diisopropyl ether and 1,2-dimethoxyethane; alcohol-type solvents such as methanol, ethanol and propyl alcohol; aliphatic hydrocarbon-type solvents such as heptane, hexane, cyclohexane and methylcyclohexane; ester-type solvents such as ethyl acetate, isopropyl acetate and butyl acetate; nitrile-type solvents such as acetonitrile and propionitrile; and ketone-type solvents such as methyl isobutyl ketone. Water often is used for solvent and/or product washing.

Another object according to the present invention is a process for the manufacture of a pharmaceutically or agrochemically active compound, which comprises the process for the manufacture of a compound of formula (I), (VII) and/or (IV). In particular, the invention concerns a process for the manufacture of a pharmaceutically or agrochemically active compound, which comprises the process for the manufacture of a compound of formula (I), (VII) and/or (IV) wherein the agrochemically or pharmaceutically active compound is a compound of formula (VIII)

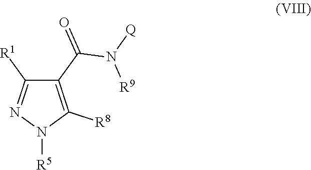

wherein R⁹ is selected from the group consisting of H, C₁-C₁₂-alkyl, C₂-C₆ alkenyl or C₃-C₈-cycloalkyl group, wherein H and C₁-C₄-alkyl are preferred, and wherein Q is an optionally substituted aryl or heteroaryl group, and wherein R¹, R⁸ and R⁹ are defined as defined before in any of the preceding aspects, embodiments or variations. R⁹ preferably is methyl, ethyl, cyclopropyl or H, wherein H is most preferred. Generally, Q is an aryl or heteroaryl group, which can be optionally substituted by one or more substituents of the group S* as defined before. More specifically, Q can be an optionally substituted aromatic carbocycle, non-aromatic or aromatic heterocyclic group, all of which can also be bi- or tricyclic, wherein one or more rings which are bound to the aromatic carbocycle or heterocyclic group can be non-aromatic. Often, Q is selected from the group consisting of phenyl, naphtalene, 1,2,3,4-tetrahydronaphthalene, 2,3-dihydro-1H-indene, 1,3-dihydroisobenzofuran, 1,3-dihydrobenzo[c]thiophene, 6,7,8,9-tetrahydro-5H-benzo[7]annulene, thiophene, furan, thiazole, thiadiazole, oxazole, oxadiazole, pyridine, pyrimidine, triazine, tetrazine, thiazine, azepine and diazepine, each of which is optionally substituted by one or more substituents of the group S* as defined before. In one aspect, Q is a group of formula Q1

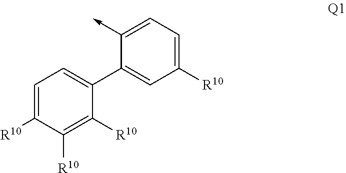

wherein each R¹⁰ is independently selected from the group consisting of hydrogen or halogen, said halogen is especially chlorine or fluorine. In particular, Q1 is the residue 3′,4′-dichloro-5-fluorobiphenyl-2-yl or the residue 3′,4′,5′-trifluorophenyl)phenyl.

In another aspect, Q is a group of formula Q2

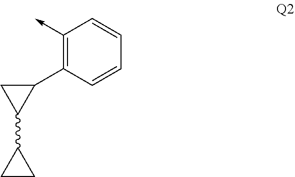

In another aspect, Q is a group of formula Q3, including all its stereoisomers:

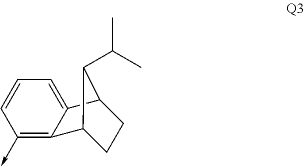

In yet another aspect, Q is a group of formula Q4

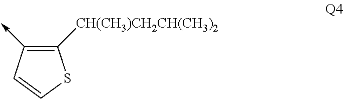

In a further aspect, Q is a group of formula Q5, including all of its stereoisomers, wherein R¹¹ is H or halogen, in particular R¹¹ is Cl.

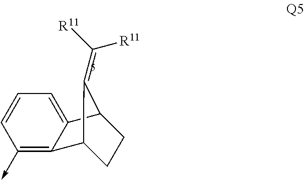

In a further aspect of the present invention, the process for the manufacture of a compound of formula (VIII) comprises the step of contacting a compound of formula (IV) with a compound of formula (IX) NHR⁹Q. Details of such a procedure are given in WO2017129759, which is incorporated by reference for all purposes. In yet another aspect of the present invention, the process for the manufacture of a compound of formula (VIII) comprises a step of converting the compound of formula (VII) into an activated carboxylic acid, preferably a carboxylic acid halide, and a step of contacting the activated carboxylic acid form of formula (VII) with a compound of formula (IX) NHR⁹Q. When the compound of formula (VII) is converted into a carboxylic acid halide, this conversion is achieved by methods known to the skilled person, for example by contacting (VII) with a halogenating agent which often is selected from the group consisting of oxalyl chloride, thionyl chloride, phosphorous trichloride, PCl₅, POCl₃ and COCl₂ and phosphorous pentachloride, Ph₃P and CCl₄ and cyanuric chloride. The carboxylic acid halide of the compound of formula (VII) is then contacted with the compound of formula (IX) NHR⁹Q, preferably in the presence of an organic base which is different from the compound of formula (IX); preferably, such a base is triethylamine, pyridine or diisopropylamine. The compound of formula (VII) can also be converted into an activated carboxylic acid form of formula (VII) by reaction with an acylating agent, wherein suitable acylating agents generally include carboxylic acid anhydrides, such as acetic acid anhydride and trifluoroacetic acid anhydride, and carboxylic acid halides, such as trifluoroacetyl chloride. In the reaction of the compound of formula (VII) with an acylating agent, presence of a base, such as, for example, triethyl amine, can be advantageous. The obtained acylated form of the compound of formula (VII) can then be contacted with the compound of formula (IX) NHR⁹Q to obtain the compound of formula (VIII). In this reaction, it can also be advantageous to have a base present which is different from the compound of formula (IX); preferably, such a base is triethylamine, pyridine or diisopropylamine. The compound of formula (VII) can also be converted into its activated form by reaction with CDI (carbonyldiimidazole). The activation of carboxylic acids with CDI, as can be applied to the compound of formula (VII) in a first step prior to the reaction with the compound of formula (IX) is described, for example, in E. K. Woodman et al, Org. Process Res. Dev., 2009, 13 (1), pp 106-113.

In a preferred aspect, the invention concerns a process for the manufacture of a pharmaceutically or agrochemically active compound, which comprises the process for the manufacture of a compound of formula (I), (VII) and/or (IV), in particular wherein the agrochemically or pharmaceutically active compound is a compound of formula (VIII), wherein the agrochemically active compound is selected from the group consisting of Sedaxane, Fluopyram, Benzovindiflupyr, Bixafen, Fluxapyroxad, Isopyrazam, Penflufen and Penthiopyrad. The processes according to the present invention can be out continuously, semi-batchwise or batchwise.

The process according to the present invention gives access to a key intermediate (I) for the manufacture of pyrazole derivatives, which in turn are key intermediates in the manufacture of agrochemically and pharmaceutically active compounds. Generally, selectivity of pyrazoles cyclization is enhanced; preparation of starting materials involving the addition of carboxylic acid halides or anhydrides such as R¹C(O)F, R¹C(O)Cl or (R¹(O)C)₂O to unsaturated intermediates, which may cause impurities, is avoided.

Should the disclosure of any patents, patent applications, and publications which are incorporated herein by reference conflict with the description of the present application to the extent that it may render a term unclear, the present description shall take precedence.

The following examples are intended to further explain the invention without limiting it.

The starting materials according to the present invention are commercially available or are obtainable through methods known to the person skilled in the art.

### EXAMPLE 1

### 4-(2-benzylidene)-1-methylhydrazinyl)-1,1,1-trichlorobut-3-en-2-one

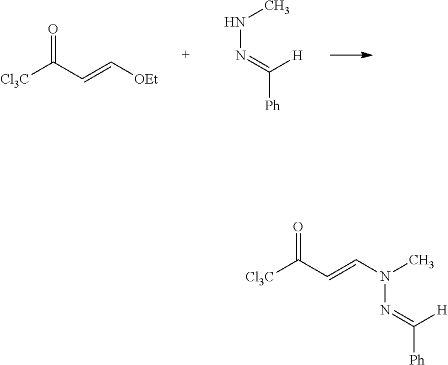

217.5 g (1 mol) 1,1,1-trichloro-4-ethoxybut-3-en-2-one (ETCBO) is dissolved in in 150 mL ethyl acetate. 141 g (1.05 mol) 1-benzylidene-2-methylhydrazine, obtained by reaction of benzaldehyde and methylhydrazine, in 150 mL ethyl acetate are added while the reaction is cooled with a water bath. After completed reaction, the reaction mixture is stirred at room temperature for 2 hours. The suspension is cooled to 0° C., filtered, the solid washed with cold ethyl acetate, and the solid is dried in vacuum.

### EXAMPLE 2

### Vinamidinium Salt (a), Starting from 4-(2-benzylidene)-1-methylhydrazinyl)-1,1,1-trichlorobut-3-en-2-one

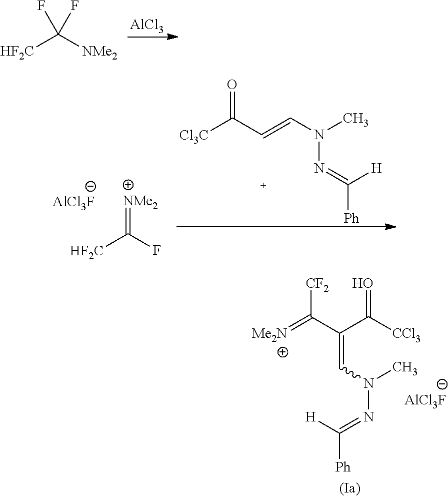

2 g AlCl₃ (68.7 mmol) are placed into a Teflon flask under nitrogen. 50 mL of anh. dichloromethane were added. The mixture was cooled to 0° C. 10.2 g 1,1,2,2-tetrafluoro-N,N-dimethylethanamine (TFEDMA) were carefully added over 10 minutes. After 1 hour at 0° C., 20 g 4-(2-benzylidene)-1-methylhydrazinyl)-1,1,1-trichlorobut-3-en-2-one from example 1 were added in portions at 0° C. under nitrogen flux and stirring. After completed addition, the mixture was allowed to warm to room temperature, and then heated under nitrogen at 50° C. for 5 hours. The mixture was cooled to room temperature. A control by ¹H-NMR in CD₃CN showed 92% conversion.

### EXAMPLE 3

### 2,2,2-trichloro-1-(3-(difluoromethyl)-1-methyl-1H-pyrazol-4-yl)ethan-1-one

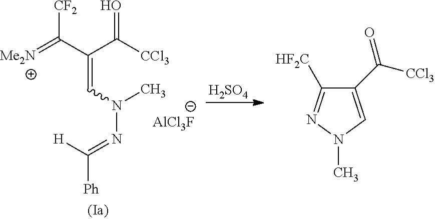

To the mixture of example 2, 3, 21 g H₂SO₄ (d=1.84, 0.5 eq) was added, and the mixture was stirred at room temperature for 30 minutes. The mixture was quenched on ice, the layers were separated and the aqueous layer extracted with dichloromethane. The layers were combined. The GC-MS showed the presence of 2,2,2-trichloro-1-(3-(difluoromethyl)-1-methyl-1H-pyrazol-4-yl)ethan-1-one.

### EXAMPLE 4

### 3-(difluoromethyl)-1-methyl-1H-pyrazole-4-carboxylic Acid

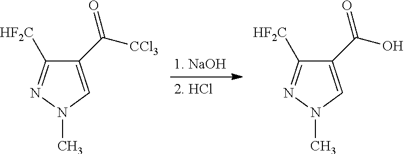

The organic phase of example 3, containing 2,2,2-trichloro-1-(3-(difluoromethyl)-1-methyl-1H-pyrazol-4-yl)ethan-1-one, was treated with 15% aq. NaOH (1.2 eq) for 1 hour at 50° C. The mixture was cooled to room temperature. The layers were separated, the aqueous layer was washed with dichloromethane, and the aqueous layer was acidified at 0° C. with 7.1 mL HCl (37% in water). After 15 minutes at 0° C., the mixture was filtered, the solid was rinsed with cold water and dried overnight in vacuum. The overall yield, starting from example 2, was 72.4%.

### EXAMPLE 5

### Vinamidinium Salt (Ib), Starting from 4-(2-benzylidene)-1-methylhydrazinyl)-1,1,1-trichlorobut-3-en-2-one

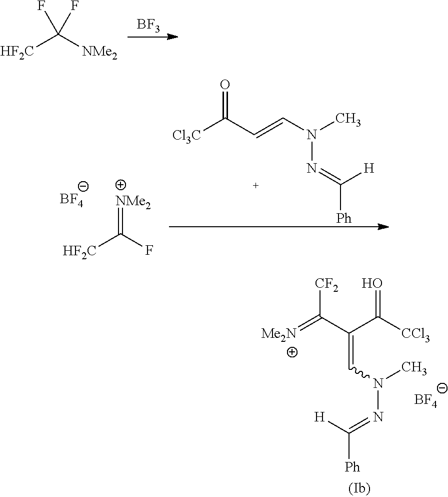

In a Teflon flask, 7.36 g BF₃.Et₂O (1.06 eq, d=1.15, 51.9 mmol) are mixed with a solution of 7.69 g (1.06 eq, 51.9 mmol) of 1,1,2,2-tetrafluoro-N,N-dimethylethanamine (TFEDMA) in 50 mL anh. dichloromethane under nitrogen at room temperature. After 1 hour at room temperature, 15 g (1.0 eq, 49.1 mmol) 4-(2-benzylidene)-1-methylhydrazinyl)-1,1,1-trichlorobut-3-en-2-one from example 1 were added in portions at room temperature under nitrogen flux and stirring. After completed addition, the mixture was allowed to warm to room temperature, and then heated under nitrogen at 50° C. for 1.15 hours. The mixture was cooled to room temperature. A control by ¹H-NMR in CD₃CN showed full conversion.

### EXAMPLE 6

### 2,2,2-trichloro-1-(3-(difluoromethyl)-1-methyl-1H-pyrazol-4-yl)ethan-1-one

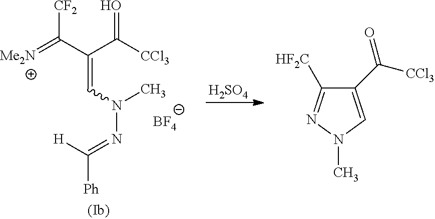

To the mixture of example 5, 7.8 mL H₂SO₄ (d=1.84, 3 eq) was added together with 2.7 mL H₂O, and the mixture was stirred at 70° C. for 30 minutes. Another 1 eq H₂SO₄ was added, and the mixture stirred for 1 hour at 70° C., then for 1 hour at room temperature. A ¹H-NMR in CD₃CN showed full cyclization of (Ib). The mixture was quenched on ice, the layers were separated and the aqueous layer extracted with dichloromethane. The layers were combined. The GC-MS showed the presence of 2,2,2-trichloro-1-(3-(difluoromethyl)-1-methyl-1H-pyrazol-4-yl)ethan-1-one.

### EXAMPLE 7

### 3-(difluoromethyl)-1-methyl-1H-pyrazole-4-carboxylic Acid

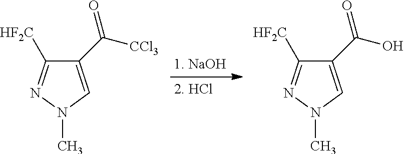

The organic phase of example 6, containing 2,2,2-trichloro-1-(3-(difluoromethyl)-1-methyl-1H-pyrazol-4-yl)ethan-1-one, was treated with 15% aq. NaOH (15.7 mL, 1.2 eq) for 1 hour at 50° C. The mixture was cooled to room temperature. The layers were separated, the aqueous layer was washed with dichloromethane, and the aqueous layer was acidified at 0° C. with 5.3 mL HCl (37% in water). After 15 minutes at 0° C., the mixture was filtered, the solid was rinsed with cold water and dried overnight in vacuum. The overall yield, starting from example 5, was 67%.

### EXAMPLE 8

### Bixafen(N-(3′,4′-dichloro-5-fluorobiphenyl-2-yl)-3-(difluoro-methyl)-1-methyl-1H-pyrazole-4-carboxamide)

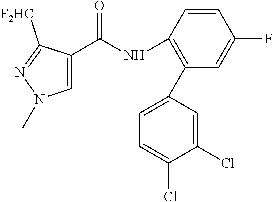 [SMILES: Cn1cc(C(=O)Nc2ccc(F)cc2-c2ccc(Cl)c(Cl)c2)c(C(F)F)n1]

5.0 g (18 mmol) 2,2,2-trichloro-1-(3-(difluoromethyl)-1-methyl-1H-pyrazol-4-yl)ethanone, and 3′,4′-dichloro-5-fluorobiphenyl-2-amine (4.6 g, 18 mmol) are mixed and diluted with 10 ml toluene. To this solution 1,1,3,3-tetramethylguanidine (TMG, 0.2 eq) is added and the mixture is stirred at room temperature for 16 hours. The volatiles of the resulting yellow suspension are evaporated and the residue is triturated with cold water. Solids are filtered, washed with water and dried yielding crude Bixafen.

### EXAMPLE 9

### Fluxapyroxad(3-(difluoromethyl)-1-methyl-N-(3′,4′,5′-trifluoro-biphenyl-2-yl)-1H-pyrazole-4-carboxamide)

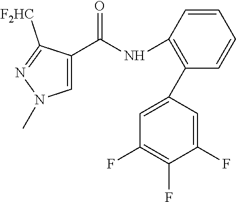 [SMILES: Cn1cc(C(=O)Nc2ccccc2-c2cc(F)c(F)c(F)c2)c(C(F)F)n1]

Fluxapyroxad is obtained using the procedure of example 8, wherein 3′,4′,5′-trifluorobiphenyl-2-amine is used instead of 3′,4′-dichloro-5-fluorobiphenyl-2-amine.

### EXAMPLE 10

### Sedaxane(N-(2-(bi(cyclopropan)-2-yl)phenyl)-3-(difluorome-thyl)-1-methyl-1H-pyrazole-4-carboxamide)

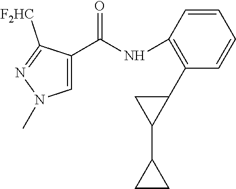 [SMILES: Cn1cc(C(=O)Nc2ccccc2C2CC2C2CC2)c(C(F)F)n1]

Sedaxane is obtained using the procedure of example 8, wherein 2-(bi(cyclopropan)-2-yl)aniline is used instead of 3′,4′-dichloro-5-fluorobiphenyl-2-amine.

### EXAMPLE 11

### 3-(difluoromethyl)-1-methyl-1H-pyrazole-4-carbonylchloride

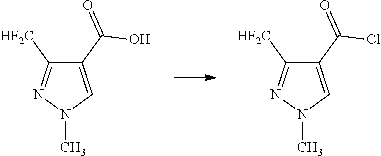

3-(difluorochloromethyl)-1-methyl-1H-pyrazol-4-carboxylic acid obtained by example 7 or 4 is treated with oxalyl chloride (1.25 eq) in toluene, and a few drops of dimethylformamide are added. The mixture is concentrated under reduced pressure to yield the carboxyl chloride.

### EXAMPLE 12

### Bixafen (N-(3′,4′-dichloro-5-fluorobiphenyl-2-yl)-3-(difluorome-thyl)-1-methyl-1H-pyrazole-4-carboxamide)

(1.3 mmol) 3′,4′-dichloro-5-fluoro-1,1′-biphenyl-2-amine and (1.56 mmol) 3-(difluorochloromethyl)-1-methyl-1H-pyrazol-4-carboxylic acid chloride obtained by Example 11 are solved in 6 ml tetrahydrofuran and mixed with 2.6 mmol triethylamine. The mixture is stirred for 16 h at 60° C. The mixture is concentrated and chromatographed on silica using cyclohexane/acetic acid ethyl ester to yield Bixafen.

### EXAMPLE 13

### Fluxapyroxad (3-(difluoromethyl)-1-methyl-N-(3′,4′,5′-tri-fluorobiphenyl-2-yl)-1H-pyrazole-4-carboxamide)

Fluxapyroxad is obtained using the procedure of example 12, wherein 3′,4′,5′-trifluorobiphenyl-2-amine is used instead of 3′,4′-dichloro-5-fluorobiphenyl-2-amine.

### EXAMPLE 14

### Sedaxane (N-(2-(bi(cyclopropan)-2-yl)phenyl)-3-(difluorome-thyl)-1-methyl-1H-pyrazole-4-carboxamide)

Sedaxane is obtained using the procedure of example 12, wherein 2-(bi(cyclopropan)-2-yl)aniline is used instead of 3′,4′-dichloro-5-fluorobiphenyl-2-amine.

1. A process for manufacturing a compound according to formula (I), the process comprising reacting a compound of formula (II) and a compound of formula (III) 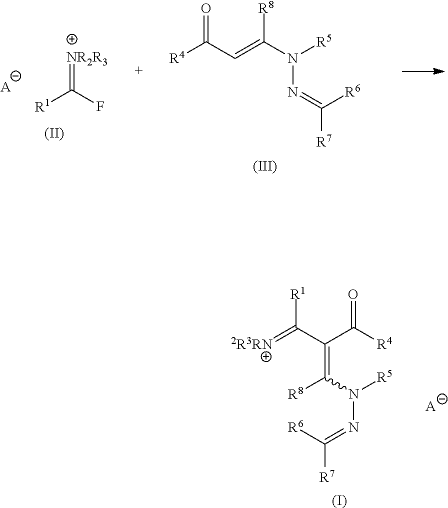
wherein
R¹ is selected from the group consisting of C₁₋₄ alkyl groups which is substituted by at least one halogen atom,
R² and R³ are independently selected from the group consisting of C₁-C₁₂-alkyl C₃-C₁₀-cycloalkyl, aryl, and aralkyl groups, each of which is optionally substituted, or wherein R² and R³ together with the nitrogen atom to which they are bound form an optionally substituted 5- to 10-membered heterocyclic radical which, in addition to the nitrogen atom to which they are attached, may contain a further 1, 2 or 3 heteroatoms selected from the group consisting of O, N and S as ring members,
R⁴ is selected from the group consisting of CF₃, CCl₃ and CBr₃,
R⁵ is selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted,
R⁶ and R⁷ each independently are selected from the group consisting of H, C₁-C₁₂-alkyl, C₂-C₆ alkenyl, C₃-C₁₀-cycloalkyl, C₂₋₁₂ alkynyl, aryl, heteroaryl or aralkyl group, each of which is optionally substituted wherein at least one of R⁶ and R⁷ is different from H,
R⁸ is selected from the group consisting of H, X′, COOR′, OR′, SR′, C(O)NR′₂, wherein the groups R′ are selected independently in C(O)NR′₂ where R′ selected from the group consisting of hydrogen, C₁-C₁₂-alkyl, CN, C₂-C₆ alkenyl, aryl, cycloalkyl, aralkyl, heteroaryl, each of which is optionally substituted, and wherein X′ is a halogen atom,
and wherein A⁻ is selected from the group consisting of Cl⁻, BF₄⁻, PF₆⁻, SbF₆⁻, AlCl₃F⁻ and AlCl₄⁻.

2. The process according to claim 1, wherein R¹ is a methyl group substituted by at least one halogen atom.

3. The process according to claim 1, wherein A⁻ is AlCl₃F⁻, BF₄⁻ or AlCl₄⁻.

4. The process according to claim 1, wherein R⁴ is CCl₃.

5. The process according to claim 1, wherein R⁸ is H or X′.

6. The process according to claim 1, wherein R⁵ is selected from the group consisting of H, CH₃ and optionally substituted benzyl.

7. The process according to claim 1, wherein R⁶ and R⁷ each independently are selected from the group consisting of H, C₁-C₁₂-alkyl and aryl, each of which is optionally substituted, wherein at least one of R⁶ and R⁷ is different from H.

8. A process for the manufacture of a compound of formula (IV), which comprises the process according to claim 1, and which further comprises a step wherein the compound of formula (I) is contacted with an acid 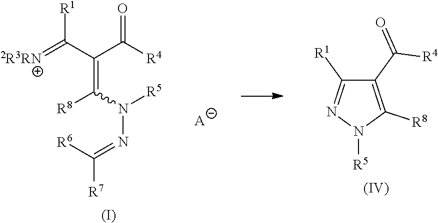
wherein R¹, R², R³, R⁴, R⁵, R⁶, R⁷, R⁸ and A⁻ are defined as in claim 1.

9. The process according to claim 8, wherein the acid is selected from the group consisting of CH₃COOH, H₂SO₄, HNO₃, H₂PO₄, KHSO₄, NaH₂PO₄, HCl, CF₃SO₃H, CF₃COOH and CH₃COONa.

10. A process for the manufacture of a compound of formula (IV), which comprises the process according to claim 1, and which further comprises a step of contacting the compound of formula (I) with water in the presence of an acid or a base under reaction conditions which allow the compound of formula (V) to be formed, followed by a step wherein the compound of formula (V) is contacted with an acid to form the compound of formula (IV), wherein R¹, R², R³, R⁴, R⁵, R⁶, R⁷, R⁸ and A⁻ are defined as in claim 1,

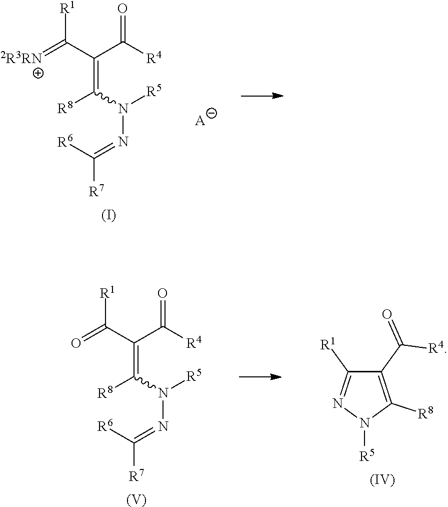

11. A process for the manufacture of a compound of formula (VII), which comprises the process according to claim 8, and which further comprises a step wherein the compound of formula (IV) is contacted with a base, 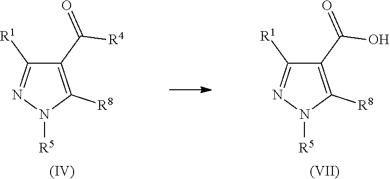
wherein
R¹ is selected from the group consisting of C₁₋₄ alkyl groups which is substituted by at least one halogen atom,
R⁴ is selected from the group consisting of CF₃, CCl₃ and CBr₃,
R⁵ is selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted,
R⁸ is selected from the group consisting of H, X′, COOR′, OR′, SR′, C(O)NR′₂, wherein the groups R′ are selected independently in C(O)NR′₂ where R′ selected from the group consisting of hydrogen, C₁-C₁₂-alkyl, CN, C₂-C₆ alkenyl, aryl, cycloalkyl, aralkyl, heteroaryl, each of which is optionally substituted, and wherein X′ is a halogen atom.

12. A process for the manufacture of a pharmaceutically or agrochemically active compound, which comprises the process according to claim 11.

13. The process according to claim 12, wherein the agrochemically or pharmaceutically active compound is a compound of formula (VIII) 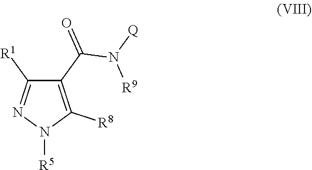
wherein R⁹ is selected from the group consisting of H, C₁-C₁₂-alkyl, C₂-C₆ alkenyl or C₃-C₈-cycloalkyl group, and wherein Q is an optionally substituted aryl or heteroaryl group, and wherein R¹, R⁸ and R⁹ are as defined.

14. The process according to claim 13, which comprises contacting the compound of formula (IV) with a compound of formula (IX), NHR⁹Q, or which comprises converting the compound of formula (VII) into an activated carboxylic acid, and contacting the activated carboxylic acid form of formula (VII) with a compound of formula (IX), NHR⁹Q.

15. The process according to claim 12, wherein the agrochemically active compound is selected from the group consisting of Sedaxane, Fluopyram, Benzovindiflupyr, Bixafen, Fluxapyroxad, Isopyrazam, Penflufen and Penthiopyrad.

16. The process according to claim 2, wherein R¹ is CHF₂ or CF₃.

17. A process for the manufacture of a compound of formula (VII), which comprises the process according to claim 10, and which further comprises a step wherein the compound of formula (IV) is contacted with a base, 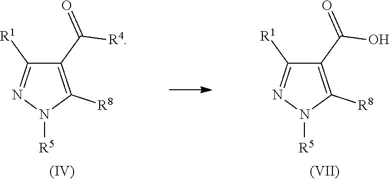
wherein
R¹ is selected from the group consisting of C₁₋₄ alkyl groups which is substituted by at least one halogen atom,
R⁴ is selected from the group consisting of CF₃, CCl₃ and CBr₃,
R⁵ is selected from the group consisting of C₁-C₁₂-alkyl, C₂-C₆ alkenyl, C₃-C₁₀-cycloalkyl, C₂₋₁₂ alkynyl, aryl, heteroaryl and aralkyl groups, each of which is optionally substituted,
R⁸ is selected from the group consisting of H, X′, COOR′, OR′, SR′, C(O)NR′₂, wherein the groups R′ are selected independently in C(O)NR′₂ where R′ selected from the group consisting of hydrogen, C₁-C₁₂-alkyl, CN, C₂-C₆ alkenyl, aryl, cycloalkyl, aralkyl, heteroaryl, each of which is optionally substituted, and wherein X′ is a halogen atom.

18. The process according to claim 13, wherein R⁹ is selected from the group consisting of H and C₁-C₄-alkyl.

19. The process according to claim 14, wherein the activated carboxylic acid is a carboxylic acid halide.
